(19) United States
(12) Patent Application Publication
(10) Pub. No.: US 2013/0079295 A1
(43) Pub. Date: Mar. 28, 2013

(54) TREATMENT OF CARTILAGE RESORPTION

(71) Applicants: Morten A. Karsdal, Kobenhavn (DK); Per Qvist, Klampenborg (DK); Qinlong Zheng, Edmond, OK (US)

(72) Inventors: Morten A. Karsdal, Kobenhavn (DK); Per Qvist, Klampenborg (DK); Qinlong Zheng, Edmond, OK (US)

(73) Assignee: NORDIC BIOSCIENCE A/S, Herlev (DK)

(21) Appl. No.: 13/695,021

(22) PCT Filed: Apr. 27, 2011
(86) PCT No.: PCT/EP2011/056686

§ 371 Date: Dec. 11, 2012

(30) Foreign Application Priority Data
Apr. 28, 2010 (EP) 1007071.2

(51) Int. Cl. A61K 36/488

(52) U.S. Cl. CPC A61K 36/488
USPC 514/27

(57) ABSTRACT

Excessive articular cartilage degradation is treated or prevented by administration to a mammal such as a human Pueraria lobata (Kudzu) root or an extract thereof.

The present invention relates to the use of Radix Pueraria lobata (also known as Kudzu root) in treatment by therapy or prophylaxis of excessive cartilage resorption, including treating osteoarthritis to produce a reduction in articular cartilage degradation or as a preventive against excessive cartilage degradation which eventually could lead to impairment of the structural integrity of the cartilage compartment, e.g. in the joints.

Radix Puerariae Lobatae (Gegen) is the dry radix part of Pueraria lobata (Willd), Ohwi. The central producing areas of Radix Puerariae Lobatae are Hunan, Guangdong, Zhejiang and Henan province of China. The plant is perennial liane or vine with a length of 10 meters. The roots are collected both in autumn and winter, cut into thick pieces and can be used after drying.

Pueraria lobata (Willd), Ohwi, has a related plant which name is Pueraria thomsonii Benth. The roots of Pueraria lobata (Wild), Ohwi and Pueraria thomsonii (Benth) have been officially recorded in all editions of Chinese Pharmacopoeia under the same monograph “Gegen” (Radix Puerariae, RP). However, in its 2005 edition, the two species were separated into both individual monographs, namely “Gegen” (Radix Puerariae Lobatae, RPL) and “Fenge” (Radix Puerariae Thomsonii, RPT). In composition, Gegen contain lots of fibre while Fenge is abundant in faecula. Both of them can be used as food and medicine.

The genus Pueraria includes five species that are referred to under the name Kudzu and these are P. edulis, P.

lobata, P. Montana, P. phaseoloides and P. thomsonii. Other plants in the genus include P. mirifica, P. omeiensis, P. peduncularis, P. tuberosa and P. wallichii.

Kudzu root has a long tradition of use in Traditional Chinese Medicine (TCM) as an active component in complex mixtures proposed for use in treating a range of conditions, not all of which are easily translated into conventional Western medical terminology.

JP-A-2007-186457 discloses Pueraria root as one of many materials acting as a tryptase activity inhibitor for treating a wide range of conditions which include rheumatoid arthritis (RA). The theory is advanced with reference to Gruber et al. J. Clin. Invest. 1989 Nov 84 (5): 1657-62 that mast cell tryptase present in rheumatoid synovium activates MMP3 and that this is active in destruction of cartilage by collagenase in RA. No evidence of the effect of Kudzu root is presented.

However, several studies in humans have demonstrated that MMP-3 inhibition does not protect articular cartilage from degradation.

CN 101002908 discloses the use of Kudzu root (according to a published extract on Espacenet) for treating rheumatoid arthritis when used in a mixture of 17 Chinese medicinal materials in an externally applied emplastrum. However, the term ‘rheumatoid arthritis’ is not used in the original specification, which instead refers to ‘arthritis with fixed pain caused by dampness’. This would seem to be more of a folk description of pain than a medical term. Many other pain inducing conditions are also referred to. Possibly all that is being taught is pain relief and even this is not necessarily an effect of the Kudzu root.

CN1115160 describes (according to its Espacenet abstract) a medicine including Pueraria root for treating conditions including both rheumatoid and osteoarthritis. Again however, this would seem to be a translation of folk disease description into technical Western medical terminology which is not justified. The document actually speaks of dispelling rheumatism and treating enlarged joints. It is not clear that it really teaches relevance to rheumatoid arthritis or osteoarthritis. The complex herbal preparation is described as improving blood flow to cause muscles and joints to relax. However the role of Kudzu in this is unclear as Kudzu root is said to be beneficial to spleen and stomach.

CN101095845 similarly teaches a complex composition of many herbal and other ingredients including Kudzu root, which according to the Espacenet abstract is for treating diseases including arthritis, but again this would seem to be an over technical translation of a reference to rheumatism. The role of Kudzu root is said to be to expel pathogenic factors from muscle and skin.

Similarly CN1090512 describes a poultice containing several herbal medicines including Kudzu root which is for treating hyperosteogeny but also is good for rheumatism, rheumatoid arthritis and muscle strains, acting by improving blood flow.

Again, CN101391050 according to its Espacenet abstract discloses a Chinese traditional medicine remedy comprising many herbal ingredients including Kudzu root for relieving inflammations and arthritis, but it is not clear that the specification actually mentions arthritis. It teaches that the composition has the effect of improving blood circulation and relieves pain, particularly in the neck and shoulder.

CN1506052 seems to disclose a composition containing in combination genetic material from a Gram-negative coccus referred to as ‘Factor X’ and ‘Pueraria lobata juice’ as being useful in treating joint pain. However, this would not appear to be an enabling disclosure due to the obscure explanation of what is ‘Factor X’. It is not indicated in what way the composition acts to relieve arthritis.

Some scientific studies on the effects of Pueraria species have been reported. Manonai et al investigated estrogen like effects of P. mirifica on cytologic and urodynamic parameters in rats, finding that P. mirifica indeed showed an estrogen like effect.

Urasopon et al studied preventative effects of P. mirifica on bone loss in ovariectomised rats, finding that bone loss was dose dependently prevented in a manner comparable with that obtained with ethinylestradiol. However, Weaver et al reported that Kudzu root did not significantly reduce net bone resorption in a small trial on healthy postmenopausal women.

Tanko et al, having reviewed the literature conclude that estrogen may have a protective effect on articular cartilage in chondrocyte mediated degradation of the articular cartilage in explant culture as shown by a dose dependent reduction in the CTX-II cartilage degradation marker produced by estradiol.

Si-Na Kim et. al. suggested that Pueraria root extracts might be a good herbal medicine for treating osteoarthritis based on a finding that such an extract showed strong inhibitory efficacy against cytokine-induced proteoglycan degradation, prostaglandin E₂ production, nitric oxide production, and matrix-metalloproteinase expression in mouse macrophages and rabbit articular chondrocytes.

We have now unexpectedly discovered that the root of Pueraria lobata is effective to reduce loss of type II collagen. At this stage, the mechanism behind this action is unknown.

Accordingly, the invention provides a composition for the treatment by therapy or prophylaxis of excessive articular cartilage degradation comprising Pueraria lobata root or extracted mixture of active components thereof formulated for administration to a mammal.

The composition may be for the treatment by therapy of excessive articular cartilage degradation in the absence of joint inflammation.

The composition may be formulated for pharmaceutical administration but may also be formulated for use as a food ingredient or food supplement. Thus, the composition may take the form of a foodstuff enriched in the active materials, e.g. a food bar.

Suitably, said composition comprises all or essentially all of the solids components of a solvent extract of said root.

However, such solids may be partially purified to increase the active content thereof. Preferably, said partial purification increases the concentration in said solids of flavonoids and/or isoflavones.

Preferably, said solvent extract is an ethanol extract. Alternatively, said solvent extract could be a methanol extract or other suitable organic solvent extract. Also, said composition could comprise an aqueous extract of said root. A solvent extract may be used directly as the pharmaceutical composition without isolation of solids therefrom or may be subject to solvent removal to leave said solids, which before or after optional further purification.

Said solids components may be obtained by partial purification of a said solvent extract, for instance as by passage through a column of macroporous resin. Active compounds will be concentrated in this instance by discarding the run through from the column and eluting active compounds retained on the column therefrom.

More generally, an initial solvent extract may be partially purified to increase the concentration within the solids fraction of compounds active in reducing cartilage loss. In the absence of knowledge as yet of which compounds are active, such purification must be empirically guided. Thus, a candidate purification method may be tested by determining whether the purified extract is more or less active in a test of its efficacy in preventing CTX-II liberation conducted as per Example 2 below.

A suitable method may include capturing active compounds on a suitable chromatography medium which allows certain non-active materials to pass through, from which medium the active compounds may then be eluted. Macroporous resins, especially AB-8 resins are suitable for this purpose.

The extracted material may be administered in any conventional solid or liquid formulation form, for instance in tablets or capsules or other forms for oral administration. Most preferably however, it is provided as an ingredient in a foodstuff.

Suitable dosage rates in terms of extract dry matter include from 12 mg/kg body weight to 1.2 g/kg body weight, e.g. from 60 to 250 mg/kg, e.g. about 120 mg/kg in humans.

Diseases in which loss of type II collagen is a feature include osteoarthritis, against which the compositions will be useful as a preventative or as a treatment.

The extraction process and any subsequent purification may be such as to obtain isoflavones compounds present in the root. Isoflavones (including isoflavones glycosides) known to be present in such extracts include diadzin, diadzein, genistein, puerarin and biochanin A. Preferably, some or more preferably all of these are present in the composition.

When selecting chromatography fractions enriched in isoflavones or flavonoids for inclusion in a partially purified extract, one may make use of the colour changing reaction of such compounds with FeCl₃ as a convenient marker for their presence.

The invention will be further described and illustrated by the following examples in which reference is made to the accompanying drawings in which:

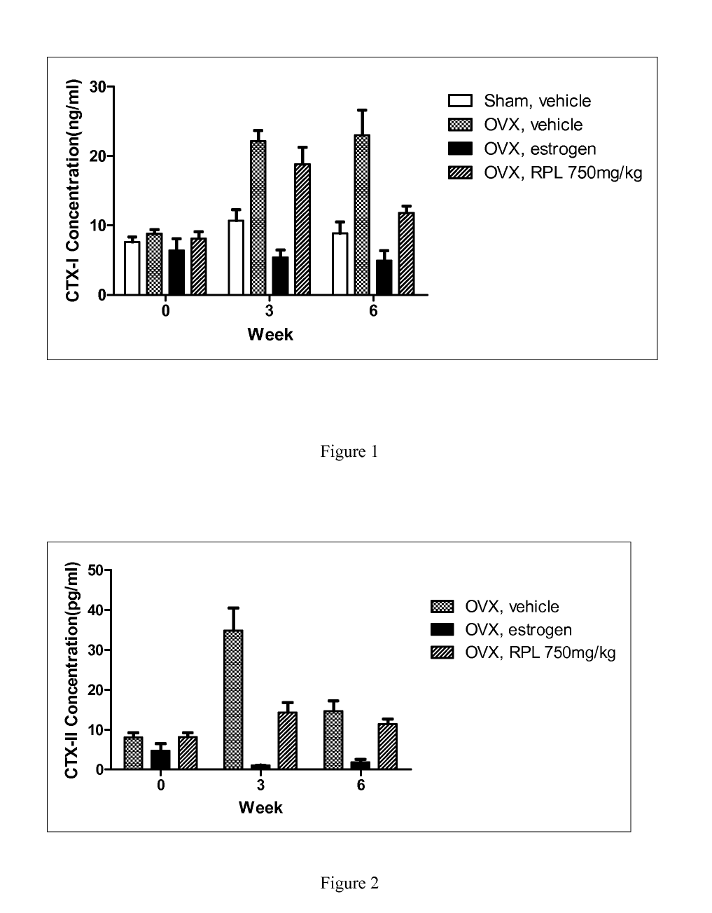

FIG. 1 shows the results of bone resorption measurements made in Example 2;

FIG. 2 shows the results of cartilage resorption measurements made in Example 2;

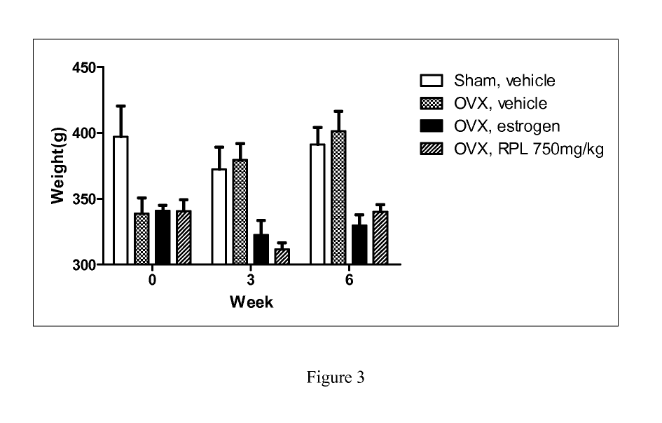

FIG. 3 shows the results of body weight measurements made in Example 2;

### EXAMPLE 1

### Extraction of P lobata Root

Root was comminuted and weighed.

### Extraction:

The pieces were put into a round flask filling less than ⅗ of volume of the flask. Eight times the volume of the powder material of 70% ethanol was added to the flask. The flask was connected to a condenser, and heated at 8° C., for 3 hours twice. The liquid extract was filtered by filter gauze to get clear liquid which was concentrated by rotary evaporator at 50° C. to produce a concentrated extract having a density around 1.1-1.2 g/cm³).

### Purification:

The concentrated extract was diluted with water to a solids concentration corresponding to the extractable content of 0.26˜0.28 g of raw material per ml of water and a pH value of 5˜6. This calculated concentration was used for the estimation of loading volume of the solution to an Ab-8 macroporous adsorption resin column. The diluted extract was purified on the AB-8 to further enrich the active ingredients of the extract and to get rid of some of impurity substances. The run through was discarded and the active ingredients of the extract in water solution were retained in AB-8 resin column and were be eluted with 70% ethanol.

The aqueous solution was absorbed by the AB-8 resin at 2 ml/min, and then desorbed using 70% ethanol at 2 ml/min; the volume of 70% ethanol was 6 times the volume of resin.

We collected the eluate in fractions, and tested for reactivity with 1% FeCl₃. Fractions changing colour from yellow to dark green indicating anti-oxidant character were collected. The 70% ethanol elution was rotary evaporated, and concentrated under low pressure at 50° C. to produce a concentrated extract of which the density was 1.1-1.2 g/cm³ which was put into flasks or plates, and frozen at −20° C. Then the flasks or plates were put in low temperature vacuum freeze dryer to produce a dry powder. The yield of solid drug/Kg of raw material varied from 50 to 100 g between lots.

### EXAMPLE 2

### Effect of Extracts on Ovariectomised Rats

An OVX screening study was carried out to evaluate the effects of TCM extracts on bone loss and cartilage degradation when given by oral gavage, to ovariectomised 6-month old Sprague-Dawley rats for 6 consecutive weeks.

The study consisted of different groups. Each group contains 10 rats and was treated with different test articles for 6 weeks. A sham operation group, an OVX group treated with vehicle and an OVX group treated with estrogen were also included as the study controls.

Serum samples were collected in the morning hours from each rat for biochemical marker analysis at baseline, 3 weeks and 6 weeks after an overnight fast. The body weight of each animal was recorded once a week. The animals were asphyxiated with carbon dioxide (CO2) and killed by exsanguination. The serum samples were measured by CTX-I (Type I collagen bone degradation marker) and CTX-II assay (Type II collagen cartilage degradation marker).

### 6.3 Statistical Analysis

The statistical analysis for the different group comparisons was performed with SPSS statistic software with a level of significance set at 0.05 for all tests. The data obtained from the animals was analyzed for homogeneity of variance using the Levene Median test and for normality using the Kolmogorov-Smirnov test. For the comparison of OVX vehicle control group with Sham group, parametric data were analyzed using Independent-Samples T-test, and non-parametric data were analyzed using Mann-Whitney Test. For all groups, parametric data were analyzed using Analysis of Variance (ANOVA), and the significance of intergroup differences (Vehicle Control vs Sample-treated group) was determined using Dunnet's T-test. Non-parametric data were analyzed using the Kruskal-Wallis test, and the significance of intergroup differences between the Vehicle Control and Test Article-treated groups was determined using Dunn's test.

### Results

### In Vivo Effect of TCM-based Herbal Extract on Bone Resorption

FIG. 1 shows results from a first study. Six-month-old rats were either sham-operated (Gr. 1) or ovariectomised (OVX). The OVX rats were treated orally with TCM-based herbal extract (Gr. 10; NBB-K6, 750 mg/Kg body weight, 17-beta-estradiol (Gr. 3) or vehicle (Gr. 2) once daily for 6 weeks. The dried extract was re-dissolved in water at a concentration of 120 mg/ml and given to rats daily in doses of 750 mg/kg body weight by gavage.

Serum CTX-I levels were determined as a biochemical parameter of bone resorption and measured at the baseline just before OVX operation (0 week); 3 weeks after OVX operation (3 week); and 6 weeks after OVX operation (6 week).

The average individual change in CTX-I over the 6-week treatment period was plotted for each treatment group with error bars representing standard error of mean (SEM).

OVX induced estrogen deficiency results in significantly higher levels of CTX-I after ovariectomy when compared to the sham group. This observation is in accord with the expected increase in bone turnover induced by ovariectomy. The estrogen implants are able to completely normalize CTX-I levels in the OVX rats. The extract NBB-K6 (Gr. 10) shows significant effects on the reduction of bone resorption, CTX-I levels, in the OVX rats both at week 3 and week 6 (p<0.005).

### In Vivo Effect of TCM-based Herbal Extract on Articular Cartilage Degradation

Serum CTX-II Concentration at the Different Time-points in Each Experimental Group (TCM-8 Study)

FIG. 2 shows corresponding measurements of the average level of serum CTX-II (pg/ml) over the 6-week treatment period plotted for each treatment group with error bars representing standard error of mean (SEM).

OVX induced estrogen deficiency results in significant increased levels of CTX-II at week 3 after ovariectomy compared to sham group. The estrogen treatment was able to completely normalize CTX-II levels in the OVX rats. The TCM-based herbal extract NBB-K6 showed a significant effect on the reduction of the cartilage degradation product of CTX-II (p<0.05).

We think it unlikely that the observed result would be due to an anti-inflammatory effect as the ovariectomised rat does not exhibit joint inflammation or other inflammatory conditions. The skilled person will appreciate, that the observed suppression of circulating levels of CTX-II fragments observed in the ovariectomised rats upon treatment with the extract of RPL, is a reflection of reduced degradation of cartilage, and that this regaining of metabolic cartilage balance in an otherwise healthy animal, is unrelated to relief of any other disease stage or state. Eventually this effect will translate into a similar beneficial effect in a human being or other mammal with elevated metabolic activity in the joints, in particular in the articular cartilage. The skilled person will understand, that the observed effect on cartilage metabolism observed in the ovariectomised rat is not a reflection of inhibition of inflammation, or any other such pathological condition, but reflects that the cartilage compartment has regained normal or near normal metabolic activity levels, which if maintained over long periods of time will maintain joint function.

### Effect of TCM-based Herbal Extract on the Growth of Animal Body Weight

FIG. 3 shows the average body weight of the rats in each group over the 6 week study with error bars representing standard error of mean (SEM). Ovariectomy induced significant weight gain in the animals, reaching 18% in the OVX+vehicle group (Gr. 2) at week 6 after ovariectomy, in accordance with the established effects of deprivation of endogenous estrogen production. The corresponding changes in the OVX and estrogen treated group (Gr. 3) and the OVX and Kudzu extract treated group (Gr. 10) were −5.5%, and −2.5% respectively, which is significantly different from the OVX+vehicle control group (Gr. 2).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

Manonai J, Seif C, Bohler G, Junemann K P, Menopause, 2009 Mar-Apr, 16 (2): 350-6
Si-Na Kin, Hee-Seok Kim, et al. Inhibition of inflammatory-cytokines production and prostaglandin E2 Activity by Puerariae Radix extracts, J Korean Soc Food Sci Nutr, 2006, 35 (1), 28-34.
Tanko L B, Sondergaard B-C, Oestergaard 5, Karsdal M A, Christiansen C; Climacteric 2008, 11:4-16
Urasopon N, Hamada Y, Cherdshewarsart W, Malaivijitnond S. Maturitas 2008, Feb 20; 59 (2); 137-48
Weaver C M, Martin B R, Jackson G S, McCabe G P, Nolan J R, McCabe L D, Barnes S, Reinwald S, Boris M E, Peacock M; J Clin Endroclinol Metab 2009, Oct, 94 (10), 3798-805

1. A composition for the treatment by therapy or prophylaxis of excessive articular cartilage degradation comprising Pueraria lobata root or extracted mixture of active components thereof formulated for administration to a mammal.

2. The composition as claimed in claim 1, which is for the treatment by therapy of excessive articular cartilage degradation in the absence of joint inflammation.

3. The composition as claimed in claim 1, formulated for pharmaceutical administration or for use as a food ingredient or food supplement.

4. The composition as claimed in claim 1, wherein said composition comprises the solids components of a solvent extract of said root.

5. The composition as claimed in claim 1, comprising solids components of a solvent extract of said root partially purified to increase the active content thereof.

6. The A composition as claimed in claim 5, wherein said partial purification increases the concentration in said solids of flavonoids and/or isoflavones.

7. The composition as claimed in claim 1, wherein said solvent extract is an organic solvent or aqueous extract.

8. The composition as claimed in claim 7, wherein said solvent extract is an ethanol or methanol extract.

9. A method for the treatment by therapy or prophylaxis of excessive articular cartilage degradation comprising administering to a mammal in need thereof Pueraria lobata root or extracted mixture of active components thereof formulated for administration to a mammal.

10. The method as claimed in claim 9, comprising administration of a composition which comprises solids components of a solvent extract of said root.

11. The method as claimed in claim 10, wherein said composition comprises solids components of a solvent extract of said root partially purified to increase the active content thereof.

12. The method as claimed in claim 11, wherein said partial purification increases the concentration in said solids of flavonoids and/or isoflavones.

13. The method as claimed in claim 10, wherein said solvent extract is an organic solvent or aqueous extract.

14. The method as claimed in claim 13, wherein said solvent extract is an ethanol or methanol extract.
